(19) United States
(12) Patent Application Publication
(10) Pub. No.: US 2024/0296436 A1
(43) Pub. Date: Sep. 5, 2024

(54) SYSTEMS AND METHODS FOR AUTOMATED PRE-PAYMENT ANALYSIS OF ELECTRONIC HEALTH CARE CLAIMS

(71) Applicant: OPTUM, INC., Minnetonka, MN (US)

(72) Inventor: Karen Michele Welsh Phillips, Raleigh, NC (US)

(21) Appl. No.: 18/177,451

(22) Filed: Mar. 2, 2023

(51) Int. Cl. G06Q 20/28; G06F 16/903; G06Q 40/08

(52) U.S. Cl. CPC G06Q 20/28; G06F 16/90335; G06Q 40/08

(57) ABSTRACT

A method for prepay review of electronic health care claims includes obtaining an electronic health care claim; determining whether the electronic health care claim is eligible for prepay analysis based on an identifier for an intended recipient of the electronic health care claim; if it is determined that the electronic health care claim is eligible for the prepay analysis, then performing the prepay analysis and augmenting the electronic health care claim based on the results of the prepay analysis; and transmitting the augmented electronic health care claim to a recipient computer associated with the intended recipient.

### BACKGROUND

As part of a typical processing pipeline, most health care insurance claims (herein “health care claims”) are subject to adjudication to confirm accuracy and review for adjustments, denial, etc. Adjudication performed prior to payment (e.g., by a health care payor) may be referred to as a “pre-pay” activity, whereas adjudication performed after payment may be referred to as a “post-pay” activity. Post-pay reviews are much more costly to the payor and, thus, the bar to pursue reclamation much higher. Payors may send claims-often in batches-to third party services to perform reviews as part of the adjudication process, to assist in determining how the payor will proceed with the claim. Often, this sort of “batch processing” requires implementation within the payor's computing system, for example, to place claims in a “pending” status, send to the third party for review, and reintegrate claims back into their system. For many payors, this is a complex and cost-prohibitive customization. In addition, the computing system(s) that receive health care claims (e.g., a payor computing system) may not be configured to process claims in their native data format. For example, claims data formats are often payor or submitter (e.g., health care provider) specific.

### SUMMARY

One implementation of the present disclosure is a system including: a processor; and memory having instructions stored thereon that, when executed by the processor, cause the system to: obtain an electronic health care claim; determine whether the electronic health care claim is eligible for prepay analysis based on an identifier for an intended recipient of the electronic health care claim; if it is determined that the electronic health care claim is eligible for the prepay analysis, then: i) perform the prepay analysis, and ii) augment the electronic health care claim based on results of the prepay analysis; and transmit the augmented electronic health care claim to a recipient computer associated with the intended recipient.

Another implementation of the present disclosure is a method including: obtaining an electronic health care claim; determining whether the electronic health care claim is eligible for prepay analysis based on an identifier for an intended recipient of the electronic health care claim; if it is determined that the electronic health care claim is eligible for the prepay analysis, then: i) performing the prepay analysis, and ii) augmenting the electronic health care claim based on results of the prepay analysis; and transmitting the augmented electronic health care claim to a recipient computer associated with the intended recipient.

Yet another implementation of the present disclosure is a non-transitory computer readable medium having instructions stored thereon that, when executed by a processor, cause a device to: obtain an electronic health care claim; determine whether the electronic health care claim is eligible for prepay analysis based on an identifier for an intended recipient of the electronic health care claim; if it is determined that the electronic health care claim is eligible for the prepay analysis, then: i) perform the prepay analysis, and ii) augment the electronic health care claim based on results of the prepay analysis; and transmit the augmented electronic health care claim to a recipient computer associated with the intended recipient.

Additional features will be set forth in part in the description which follows or may be learned by practice. The features will be realized and attained by means of the elements and combinations particularly pointed out in the appended claims. It is to be understood that both the foregoing general description and the following detailed description are exemplary and explanatory only and are not restrictive, as claimed.

### BRIEF DESCRIPTION OF THE DRAWINGS

FIGS. 1A and 1B are a block diagrams illustrating various configurations of a system for prepay analysis of electronic health care claims, according to some implementations.

FIG. 2 is a detailed block diagram of the Prepay Analysis Tool of FIGS. 1A and 1B. according to some implementations.

FIG. 3 is a flow chart of a process for evaluating an electronic health care claim using the Prepay Analysis Tool of FIGS. 1A-2, according to some implementations.

FIG. 4 is a flow chart of a process for performing a prepay analysis on an electronic health care claim, according to some implementations.

FIG. 5 is a flow chart of a process for determine a status of an electronic health care claim, according to some implementations.

Various objects, aspects, features, and advantages of the disclosure will become more apparent and better understood by referring to the detailed description taken in conjunction with the accompanying drawings, in which like reference characters identify corresponding elements throughout. In the drawings, like reference numbers generally indicate identical, functionally similar, and/or structurally similar elements.

### DETAILED DESCRIPTION

In many health care claims processing pipelines, a clearinghouse connects health care providers (e.g., doctors, hospitals, health care groups, etc.) with health care payors (e.g., insurance providers) to aid in the submission of health care claims. A clearinghouse may operate a server, for example, which receives health care claims, evaluates the health care claims against some criteria, and forwards the health care claims to a payor. In some implementations, the clearinghouse may return a health care claim to a provider (e.g., without sending to a payor) to allow the provider to correct specific issues which would likely cause the payor to reject the health care claim. To this point, clearinghouses generally have a limited role in assisting with payment accuracy.

Referring generally to the figures, a system and methods for automated “prepay” review of electronic health care claims is shown, accordingly to various implementations. In one implementation, a Prepay Analysis Tool is described herein which obtains electronic health care claims, automatically performs a prepay analysis of the obtained electronic health care claims, and augments the electronic health care claims based on the results of the prepay analysis. Then, the augmented electronic health care claims can be forwarded to an intended recipient (e.g., a health care payor). In some implementations, the Prepay Analysis Tool modifies the electronic health care claim based on the results of the prepay analysis. In some implementations, the Prepay Analysis Tool is integrated with a clearinghouse computing system to facilitate in the analysis and/or modification of electronic health care claims. In some such implementations, the clearinghouse may interact with the Prepay Analysis Tool as one of the final steps before sending a claim to a payor.

As described here, a “prepay analysis” includes a plurality of checks that are performed on a health care claim to ensure accuracy. As mentioned above, prepay analyses can be performed on health care claims as part of a pre-adjudication process and prior to payment. Alternatively, “pre-pay” activity can include activities performed on or to a claim post-adjudication and pre-payment. It should be appreciated that, generally, prepay analyses are subject to payor discretion and thus may be payor specific. In other words, the check or processes associated with a prepay analysis may vary from payor to payor. Additionally, or alternatively, providers may establish standards for a prepay analysis. Commonly, a prepay analysis includes one or more of a records review, claim code editing, network repricing, or a coordination of benefit (COB) check.

A records review evaluates the claim and provider records (e.g., for the provider that submitted the claim) to identify abnormal billing activities or claim errors. Claim code editing modifies one or more fields of the health care claim to correct errors or update information. Network repricing verifies or updates pricing associated with the claim, such as by applying pre-negotiated discounts, verifying claim or line code amounts, etc. A COB check is an automated process for coordinating benefited between two or more payors for a claim. In some implementations, a COB check searches for additional payor coverage, where a claim would require determination of primacy and benefit coordination between two or more payors. It should be appreciated, however, that a prepay analysis is not limited to only these activities and that other reviews and processes can be included in the prepay analysis and are contemplated herein.

At a high level, a sender (e.g., a health care provider) may submit an electronic health care claim according to their standard procedures and/or in their normal manner, and the clearinghouse may preprocess the health care claim as usual. However, before transforming the health care claim into a payor-specific Electronic Data Interchange (EDI) interpretation and/or forwarding the health care claim to the payor, the clearinghouse may check if the health care claim is eligible for prepay analysis. If so, the clearinghouse may send the health care claim to the Prepay Analysis Tool, which performs a suitable prepay analysis and, when complete, returns one or more of a modified version of the claim, an augmented claim, or results of the prepay analysis to the clearinghouse. Alternatively, the Prepay Analysis Tool may be integrated with, or implemented on, the clearinghouse server, in which case the functionality of the Prepay Analysis Tool may be implemented by the clearinghouse itself.

In some implementations, an electronic health care claim is augmented with a unique code relating to the prepay analysis. Such a code may indicate that the prepay analysis was completed and/or may indicate edits that were applied to the claim, for example. In some aspects, this may help to reduce integration requirements and computational overhead for payors, as the code could be used to retrieve or determine more detailed information on the analysis (e.g., as there would be limited space to include this type of additional detail on a standard electronic health care claim). Additionally, a payor (e.g., the recipient of the health care claim) would have key information needed for adjudicating the claim at the same time the claim is received, which could speed up the adjudication process and even assist enhancing auto-adjudication, in many cases. For example, claims with simple review needs could be released almost immediately, whereas claims with more complex needs could be held until the prepay analysis was complete or a payor dictated release time was reached. In some implementations, the unique code is an alphanumeric sting or a selectable link, but other types of codes are contemplated herein.

The disclosed Prepay Analysis Tool can provide a number of features that worth consideration. For example, many health care claim processing systems (e.g., payor computing systems) do not have the computational resources to manage claims as prepay analyses are performed (e.g., payor system may need to toggle claims statuses), which makes some current claims management systems cost and resource prohibitive. Prepay Analysis Tool also allows for prepay analysis services to be “added-on” to claims that are already being transmitted to payor, requiring little to no additional overhead for the provider or payor. As part of the prepay analysis process, claims can be converted into a standardized format that allows for rules, edits, etc., to be applied, while also converting the claims into appropriate formats for receipt by payors. This reduces or eliminates the need for payor computing systems to perform resource-intensive conversions and data integration. Further, payor or provide computer systems to not need to perform custom data ingestion (e.g., file transfer and/or import) resulting in reduced implementation times. And, as mentioned above, prepay analysis can provide for faster claims adjudication, as claims no longer need to be sent to a payor, be pulled out of the payor's system, sent to a remote system for review, and returned to the payor. Instead, claims arrive at the payor have already been analyzed and ready for payment, allowing payors to make faster decisions regarding claims reimbursement.

### Prepay Analysis Tool

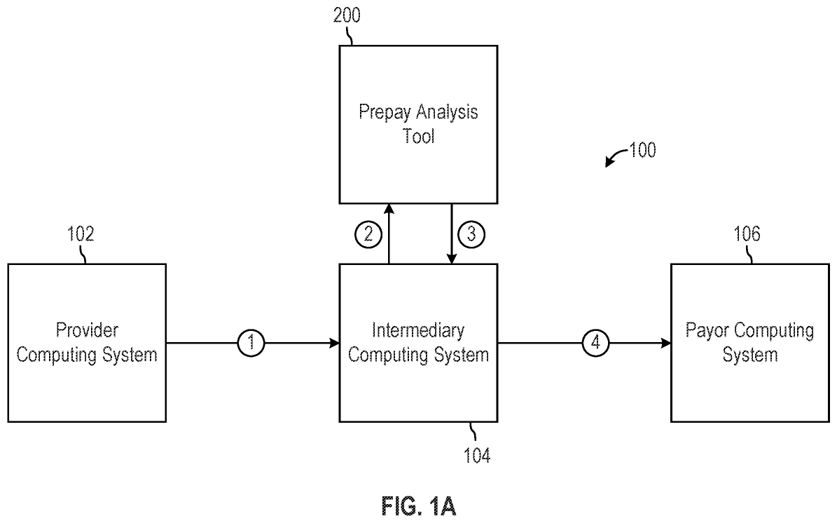

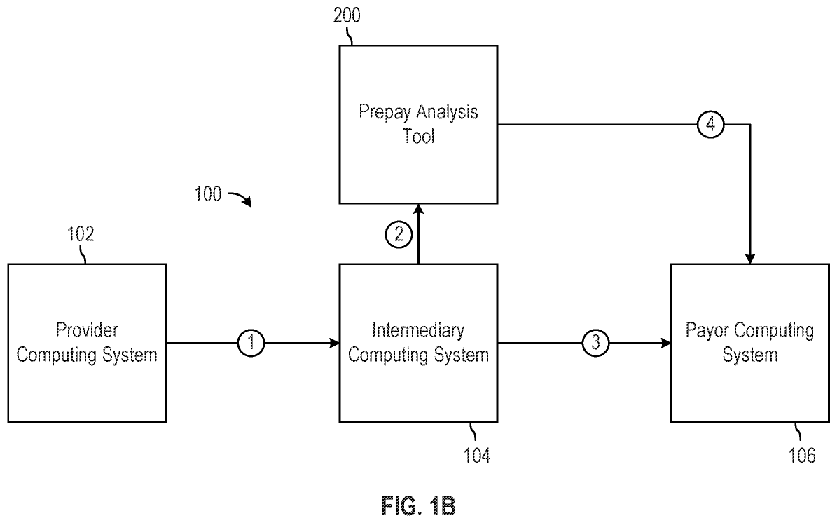

Referring now to FIGS. 1A and 1B, block diagrams illustrating various configurations of a system 100 for prepay analysis of electronic health care claims are shown, according to some implementations. System 100 is generally shown to include a provider computing system 102, an intermediary computing system 104, and a payor computing system 106. As described herein, provider computing system 102 is generally any computing system or device (e.g., a workstation, a server, etc.) that is operated by a party that submits electronic health care claims. For example, provider computing system 102 may be a computer operated by a health care provider (e.g., a doctor, a medical group, a hospital, etc.). Similarly, payor computing system 106 is generally any computing system or device (e.g., a workstation, a server, etc.) that is operated by a party that receives and/or provides payment for electronic health care claims. For example, provider computing system 102 may be a computer operated by a health care payor (e.g., a medical insurance company).

Intermediary computing system 104 may be any computing device (e.g., a server, etc.) that can intercept communications between provider computing system 102 and payor computing system 106, or that is configured to communicate with both provider computing system 102 and payor computing system 106. In some implementations, intermediary computing system 104 is a computer (e.g., a server) operated by a clearinghouse. It should be appreciated that, while only a single provider computing system 102 and payor computing system 106 are shown in the example of FIG. 1A, system 100 may include any number of provider and/or payor computing systems. For example, multiple different providers may submit health care claims to multiple different payors; thus, as generally described herein, intermediary computing system 104 may be generally configured to communicate with multiple different providers and payors. While not illustrated, it should also be appreciated that provider computing system 102, intermediary computing system 104, and/or payor computing system 106 may be communicably coupled via a direct connection or by any suitable network. For example, in some implementations, provider computing system 102, intermediary computing system 104, and payor computing system 106 are connected via the Internet using either wired or wireless connections.

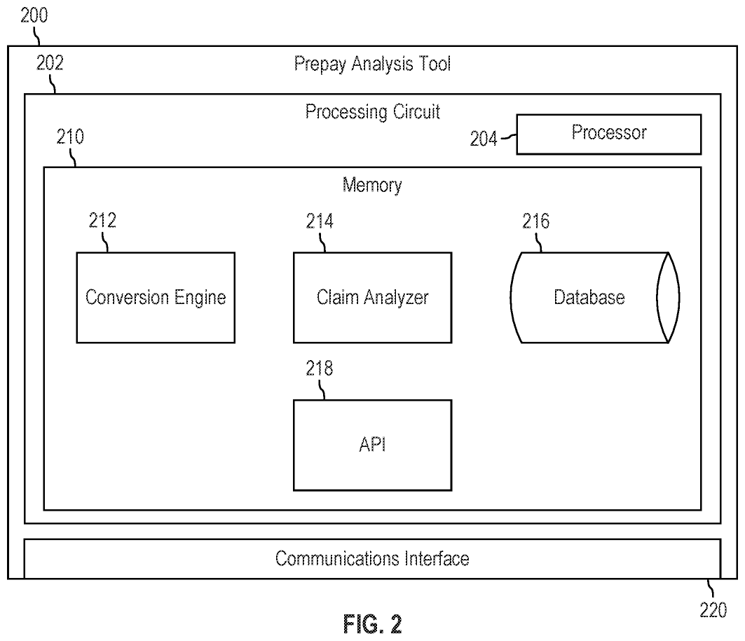

System 100 further includes a Prepay Analysis Tool 200 configured to perform a prepay analysis of electronic health care claims and, in some implementations, to edit or modify the electronic health care claims based on the results of the prepay analysis. In some implementations, Prepay Analysis Tool 200—described in greater detail below with respect to FIG. 2—is hosted on, implemented by, or includes a separate computing device. For example, Prepay Analysis Tool 200 may be hosted on its own server or workstation. In some such implementations, Prepay Analysis Tool 200 is hosted on a cloud server. In some implementations, intermediary computing system 104 communicates with Prepay Analysis Tool 200 via a network (e.g., the Internet). In some implementations, Prepay Analysis Tool 200 includes an application programming interface (API) to facilitate communications with intermediary computing system 104, and/or provider computing system 102 and payor computing system 106. For example, intermediary computing system 104 may communicate with Prepay Analysis Tool 200 via API calls. Though not illustrated in the figures, in other implementations, Prepay Analysis Tool 200 may be a component of (e.g., hosted on, embedded on, etc.) intermediary computing system 104, in which case intermediary computing system 104 may implement the functionality of Prepay Analysis Tool 200 as described herein.

Focusing on FIG. 1A, in particular, an example analysis of an electronic health care claim via system 100 is shown. In this example, provider computing system 102 is shown to submit an electronic health care claim 1) to intermediary computing system 104. Generally, a health care provider (e.g., a doctor, a hospital, etc.) can submit the electronic health care claim using their standard billing workflow. For example, the electronic health care claim can be created or populated and submitted in accordance with the health care provider's standard procedures and submitted in a normal manner (e.g., without special considerations for Prepay Analysis Tool 200). In some implementations, the electronic health care claim is submitted as an EDI 837 (“Health Care Claim”) transaction, although any other suitable format may be used.

Upon receipt of the electronic health care claim, intermediary computing system 104 may evaluate the claim to determine whether the claim is eligible for prepay analysis. If so, intermediary computing system 104 forwards (e.g., transmits) the electronic health care claim 2) to Prepay Analysis Tool 200. Alternatively, in some implementations, intermediary computing system 104 may simply forward the claim 2) to Prepay Analysis Tool 200 upon receipt without checking for prepay analysis eligibility. In such implementations, Prepay Analysis Tool 200 itself may make the prepay analysis eligibility determination. In yet other implementations, where Prepay Analysis Tool 200 is a part of intermediary computing system 104, the electronic health care claim is retained by intermediary computing system 104 (e.g., as opposed to being transmitted to Prepay Analysis Tool 200) after determining prepay analysis eligibility for further processing. In some implementations, if a claim is deemed not eligible for prepay analysis, intermediary computing system 104 or Prepay Analysis Tool 200 forwards the claim to payor computing system 106 without review or modification.

Subsequently, Prepay Analysis Tool 200 is configured to perform a prepay analysis of the electronic health care claim and return a results of the prepay analysis or a modified version of the electronic health care claim 3) to intermediary computing system 104. As described in greater detail below, the electronic health care claim may be modified by appending a code relating to the prepay analysis and/or editing or modifying one or more fields of the claim. In some implementations, the electronic health care claim is augmented with said code (e.g., in the form of a selectable link) or with a report of the prepay analysis results. In some implementations, intermediary computing system 104 alternatively receives (e.g., at (3)) the results of, or data associated with the results of, the prepay analysis and generates a modified electronic health care claim (e.g., by applying edits to the claim). For example, Prepay Analysis Tool 200 may perform the prepay analysis and transmit a report or a code to intermediary computing system 104, which intermediary computing system 104 uses to modify the electronic health care claim (e.g., by appending the code to the claim).

In some implementations, as part of or prior to the prepay analysis, Prepay Analysis Tool 200 converts the electronic health care claim from its native data format (e.g., which may be provider-specific) into a standardized data format suitable for the prepay analysis. Prior to returning the electronic health care claim, Prepay Analysis Tool 200 may optionally convert the claim back into its native data format. Alternatively, intermediary computing system 104 may perform said conversion of the claim format prior to sending the claim to Prepay Analysis Tool 200 and/or upon receipt of the augmented electronic health care claim and/or prepay analysis results from Prepay Analysis Tool 200. In any case, after receiving the augmented electronic health care claim and/or prepay analysis results, intermediary computing system 104 transmits the electronic health care claim 4) to payor computing system 106. In some implementations, intermediary computing system 104 further transmits results of the prepay analysis or otherwise makes the results of the prepay analysis available to the recipient (e.g., by remote access).

Turning to FIG. 1B, an alternate communication path for an electronic health care claim is shown. In this configuration, after receiving a submitted electronic health care claim 1), intermediary computing system 104 forwards the electronic health care claim to both Prepay Analysis Tool 200 and payor computing system 106 (2, 3). In some implementations, intermediary computing system 104 checks whether the claim is eligible for prepay analysis prior to sending the claim to Prepay Analysis Tool 200. In other implementations, Prepay Analysis Tool 200 is configured to determine prepay analysis eligibility. Prepay Analysis Tool 200 then performs the prepay analysis and sends results (e.g., a report, a modified health care claim) directly to payor computing system 106 (4). In this way, payor computing system 106 does not wait until the prepay analysis is complete to receive the electronic health claim. However, payor computing system 106 does take on the burden of integrating the results of the prepay analysis (e.g., claim modifications), if necessary.

### Prepay Analysis Tool

Referring now to FIG. 2, a detailed block diagram of Prepay Analysis Tool 200 is shown, according to some implementations. Prepay Analysis Tool 200 is shown to include a processing circuit 202 that includes a processor 204 and a memory 210. Processor 204 can be a general-purpose processor, an ASIC, one or more FPGAs, a group of processing components, or other suitable electronic processing structures. In some embodiments, processor 204 is configured to execute program code stored on memory 210 to cause Prepay Analysis Tool 200 to perform one or more operations, as described below in greater detail. It will be appreciated that, in embodiments where Prepay Analysis Tool 200 is part of another computing device (e.g., a server, a general purpose computer), the components of Prepay Analysis Tool 200 may be shared with, or the same as, the host device. To this point, Prepay Analysis Tool 200 may, in some implementations, be a web-based (e.g., online) tool that is remotely accessed by intermediary computing system 104, such as via application programming interface (API) call. In other implementations, as discussed above, Prepay Analysis Tool 200 may be a component of intermediary computing system 104.

Memory 210 can include one or more devices (e.g., memory units, memory devices, storage devices, etc.) for storing data and/or computer code for completing and/or facilitating the various processes described in the present disclosure. In some embodiments, memory 210 includes tangible (e.g., non-transitory), computer-readable media that stores code or instructions executable by processor 204. Tangible, computer-readable media refers to any physical media that is capable of providing data that causes Prepay Analysis Tool 200 to operate in a particular fashion. Example tangible, computer-readable media may include, but is not limited to, volatile media, non-volatile media, removable media and non-removable media implemented in any method or technology for storage of information such as computer readable instructions, data structures, program modules or other data. Accordingly, memory 210 can include RAM, ROM, hard drive storage, temporary storage, non-volatile memory, flash memory, optical memory, or any other suitable memory for storing software objects and/or computer instructions. Memory 210 can include database components, object code components, script components, or any other type of information structure for supporting the various activities and information structures described in the present disclosure. Memory 210 can be communicably connected to processor 204, such as via processing circuit 202, and can include computer code for executing (e.g., by processor 204) one or more processes described herein.

While shown as individual components, it will be appreciated that processor 204 and/or memory 210 can be implemented using a variety of different types and quantities of processors and memory. For example, processor 204 may represent a single processing device or multiple processing devices. Similarly, memory 210 may represent a single memory device or multiple memory devices. Additionally, in some embodiments, Prepay Analysis Tool 200 may be implemented within a single computing device (e.g., one server, one housing, etc.). In other embodiments, Prepay Analysis Tool 200 may be distributed across multiple servers or computers (e.g., that can exist in distributed locations). For example, Prepay Analysis Tool 200 may include multiple distributed computing devices (e.g., multiple processors and/or memory devices) in communication with each other that collaborate to perform operations. For example, but not by way of limitation, an application may be partitioned in such a way as to permit concurrent and/or parallel processing of the instructions of the application. Alternatively, the data processed by the application may be partitioned in such a way as to permit concurrent and/or parallel processing of different portions of a data set by the two or more computers.

Memory 210 is shown to include a conversion engine 212, which is configured to convert electronic health care claims from a native or “received” format into a standardized format suitable for conducting a prepay analysis. For example, electronic health care claims may be received in an EDI format, such as EDI 837. Accordingly, in some implementations, conversion engine 212 may convert receive electronic health care claims into another data format suitable for processing by Prepay Analysis Tool 200. In some implementations, conversion engine 212 simply extracts data from an electronic health care claim, which is used for the prepay analysis. In some implementations, conversion engine 212 converts claims from one data format to another. In some implementations, conversion engine 212 is configured to revert an electronic health care claim back to its original data format after the prepay analysis is complete. In some implementations, conversion engine 212 can convert analyzed/augmented claims into the same data format as the original claim.

Memory 210 is also shown to include a claim analyzer 214, which performs prepay analyses on electronic health care claims. As described above, a prepay analysis can include one or more of a records review, claim code editing, network repricing, a COB check, and the like. However, it should be appreciated that a prepay analysis may be provider and/or payor dependent. For example, the various checks, reviews, processes, and/or parameters for a prepay analysis may vary between different providers and/or payors. Accordingly, in some implementations, claim analyzer 214 first determines an intended or “target” recipient of the electronic health care claim (e.g., a payor that operates payor computing system 106) to inform the subsequent prepay analysis. In some such implementations, claim analyzer 214 may identify the intended recipient based on data contained within the claim (e.g., a payor ID).

In some implementations, claim analyzer 214 is configured to determine whether a received electronic health care claim is eligible for prepay analysis. In some such implementations, claim analyzer 214 makes an eligibility determination based on the intended recipient of the claim. For example, claim analyzer 214 may use a payor ID to confirm whether the payor utilizes the prepay analysis service. For a claim to be eligible for prepay analysis, the payor (e.g., claim recipient) and/or provider (e.g., claim submitter) may be required to have subscribed to a prepay analysis service (e.g., offered by an entity that operates intermediary computing system 104 and/or Prepay Analysis Tool 200). For example, one or both of a provider and a payor associated with a claim may have an agreement or contract in place to receive prepay analysis services. It should be appreciated that, in certain other implementations, intermediary computing system 104 makes prepay analysis eligibility determinations; thus, claim analyzer 214 may proceed with a prepay analysis upon receiving a claim, without separately checking for eligibility.

In some implementations, memory 210 includes a database 216 which maintains a list of payors and/or providers that are subscribed to the prepay analysis service. In some such implementations, claim analyzer 214 may query database 216 using a claim recipient identifier (e.g., a payor ID) to determine whether a claim is eligible for prepay analysis. In some implementations, database 216 further maintains a list of prepay analysis services or parameters for each payor/provider. More specifically, in some implementations, each payor and/or provider that utilizes Prepay Analysis Tool 200 can select or request a particular set of services (e.g., checks, reviews, edits, etc.) to be performed as part of a prepay analysis for claims associated with that particular payor or provider. For example, a first payor may request only a COB service, while a second payor may request a records review, claim code editing, network repricing, a COB services. In some such implementations, claim analyzer 214 may query database 216 to identify the services or parameters to be performed as part of a prepay analysis for each received claim.

In some implementations, claim analyzer 214 receives electronic health care claims in a native data format (e.g., from intermediary computing system 104) and is configured to perform prepay analyses accordingly. In some other implementations, claim analyzer 214 receives claims that have been standardized or normalized-by conversion from their native data format to a standard format-from conversion engine 212 for performing a prepay analysis. In yet other implementations, claim analyzer 214 receives data extracted from an electronic health care claim from conversion engine 212 and/or claim analyzer 214 itself extracts relevant data from the electronic health care claim, and thereby performs prepay analyses using only extracted data (e.g., instead of using the original electronic health care claim). In some implementations, claim analyzer 214 is configured to interface or communicate with one or more additional computing devices or services (e.g., cloud services) while performing a prepay analysis. For example, claim analyzer 214 may communicate with remote databases, etc.

After performing a prepay analysis for a given claim, claim analyzer 214 may generate a report of the results of the analysis and/or modify the claim. In some implementations, claim analyzer 214 generates a results report for the prepay analysis and returns the report to intermediary computing system 104 and/or forwards the report to payor computing system 106. In this manner, intermediary computing system 104 and/or payor computing system 106 can edit the claim accordingly. Alternatively, in some implementations, claim analyzer 214 performs any necessary edits to the claim. In other words, claim analyzer 214 may generate a modified electronic health care claim by editing one or more fields of the original claim based on the results of the analysis. Claim analyzer 214 may then return, to intermediary computing system 104 and/or payor computing system 106, the modified electronic health care claim.

In some implementations, rather than editing data of the health care claim, claim analyzer 214 augments the claim by appending a unique code to the claim. For example, electronic health care claims may generally be received in EDI 837 format, which does not have much space to include additional claim information. Thus, claim analyzer 214 may append a code to the original claim which provides additional data relating to the prepay analysis. In some implementations, the unique code is an alphanumeric string or a selectable link. For example, the code may be used the recipient of the claim to look up or request a prepay analysis report or data from the analysis. As another example, the code may include an embedded link to the report or a secure website/database on which the report is hosted which, when selected by a user (e.g., via a user device), automatically navigates to the website/database/report.

In some implementations, claim analyzer 214 is configured to modify or augment a data structure associated with the electronic health care claim. In this manner, claim analyzer 214 can associate the results of a prepay analysis with the health care claim without directly modifying the claim itself by instead augmenting the data structure associated with the claim. For example, claim analyzer 214 may change the data structure of a claim from an initial state (e.g., prior to the prepay analysis) to a modified or augmented state (e.g., after the prepay analysis) which includes, at least, an indication of the results of the prepay analysis and/or an indication that the prepay analysis was performed. In some implementations, claim analyzer 214 is configured to augment a claim's data structure by editing, adding, or removing fields or data. For example, claim analyzer 214 may add a report (e.g., the results of the prepay analysis) to a new data field in the data structure of the claim.

In some implementations, communications to and/or from Prepay Analysis Tool 200 are facilitated by an application programming interface (API) 218. Generally, API 218 is an interface that allows remote and/or third-party computing systems to send data to or request data from Prepay Analysis Tool 200. It will be appreciated that API 218 can be any suitable API, such as a RESTful API. Accordingly, in some implementations, Prepay Analysis Tool 200 may be cloud-based (e.g., hosted on a cloud server) and API 218 may facilitate communications with any of provider computing system 102, intermediary computing system 104, and payor computing system 106. For example, to facilitate a prepay analysis of an electronic health care claim, intermediary computing system 104 may communicate with Prepay Analysis Tool 200 via API calls. Additionally, in some implementations, Prepay Analysis Tool 200 may be accessible via a remote user device (not shown) API 218, such that a user can view or change data or otherwise control Prepay Analysis Tool 200.

In some implementations, Prepay Analysis Tool 200 is configured for file transmission or sharing via File Transfer Protocol (FTP), secure FTP (sFTP), or any other suitable file transfer technique. For example, some data/files may be communicated with Prepay Analysis Tool 200 via API 218 while others are communicated via sFTP or, in other cases, files are transferred to Prepay Analysis Tool 200 via sFTP only. In some such implementations, Prepay Analysis Tool 200 can receive batches of electronic health care claims (e.g., one or more claims) via FTP or sFTP. Additionally, or alternatively, Prepay Analysis Tool 200 can obtain electronic health care claims from one or more of provider computing system 102, intermediary computing system 104, and payor computing system 106, as generally described herein, and may transmit results of a prepay analysis for each claim to payor computing system 106 or another recipient via FTP or sFTP. In this manner, Prepay Analysis Tool 200 can share large amounts of data, or at least more data than can be included in an electronic health care claim, with external computing systems.

Prepay Analysis Tool 200 is also shown to include a communications interface 220 that facilitates communications between Prepay Analysis Tool 200 and any external components or devices (e.g., through API 218), including provider computing system 102, intermediary computing system 104, and/or payor computing system 106. Accordingly, communications interface 220 can be or can include a wired or wireless communications interface (e.g., jacks, antennas, transmitters, receivers, transceivers, wire terminals, etc.) for conducting data communications, or a combination of wired and wireless communication interfaces. In some embodiments, communications via communications interface 220 are direct (e.g., local wired or wireless communications) or via a network (e.g., a WAN, the Internet, a cellular network, etc.). For example, communications interface 220 may include one or more Ethernet ports for communicably coupling Prepay Analysis Tool 200 to a network (e.g., the Internet). In another example, communications interface 220 can include a Wi-Fi transceiver for communicating via a wireless communications network. In yet another example, communications interface 220 may include cellular or mobile phone communications transceivers.

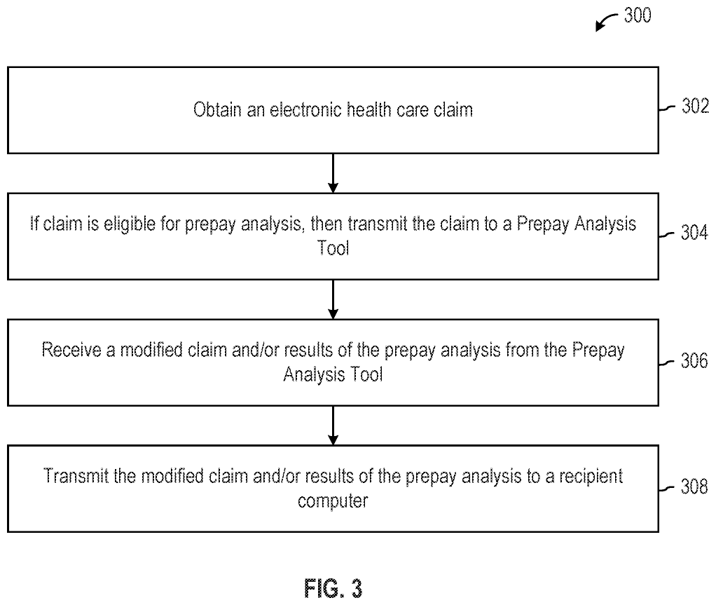

Referring now to FIG. 3, a flow chart of a process 300 for evaluating an electronic health care claim as part of a prepay analysis is shown, according to some implementations. In some implementations, process 300 is implemented by intermediary computing system 104, as described above; although, it will be appreciated that process 300, or certain steps of process 300, may be performed by Prepay Analysis Tool 200. It will be appreciated that certain steps of process 300 may be optional and, in some implementations, process 300 may be implemented using less than all of the steps. It will also be appreciated that the order of steps shown in FIG. 3 is not intended to be limiting.

At step 302, an electronic health care claim is obtained. In some implementations, the electronic health care claim (e.g., an EDI 837 “Health Care Claim”) is received from provider computing system 102 upon submission by a health care provider. In some such implementations, the provider may submit the electronic health care claim directly to intermediary computing system 104 and/or Prepay Analysis Tool 200. In some implementations, the electronic health care claim is received from payor computing system 106. For example, payor computing system 106 may forward the claim to intermediary computing system 104 and/or Prepay Analysis Tool 200 for evaluation upon receiving the original claim from provider computing system 102. In some implementations, the electronic health care claim is intercepted as it is transmitted from provider computing system 102 to payor computing system 106 (e.g., from a payor to a provider). For example, intermediary computing system 104 and/or Prepay Analysis Tool 200 may monitor communications between provider computing system 102 and payor computing system 106 to detect and intercept claims. Generally, in such implementations, the provider and payor have a previously established agreement that intermediary computing system 104 and/or Prepay Analysis Tool 200 can intercept, analyze, and/or modify claims.

At step 304, the electronic health care claim is transmitted (e.g., forwarded) to Prepay Analysis Tool 200 if it is determined that the claim is eligible for prepay analysis. Accordingly, prior to or as part of step 304, a prepay analysis eligibility determination is made. In some implementations, intermediary computing system 104 evaluates the electronic health care claim to identify a recipient of the claim (e.g., a payor) and determines whether the recipient of the claim is eligible for prepay analysis. For example, intermediary computing system 104 may determine a recipient ID and/or may determine whether the claim recipient is registered to use or has signed up for prepay analysis services. To this point, in some cases, the prepay analysis service may be a stand-alone service offered to health care providers and/or payors and, as such, only those providers/payors who have entered into a contract with the entity that operates intermediary computing system 104 and/or Prepay Analysis Tool 200 may be eligible for prepay analysis. In some implementations, rather than intermediary computing system 104 determining eligibility, claims are simply forwarded to Prepay Analysis Tool 200 after receipt, whereupon Prepay Analysis Tool 200 makes the prepay analysis eligibility determination. As mentioned above, in some implementations, Prepay Analysis Tool 200 is accessible to intermediary computing system 104 via API 218; thus, step 304 may include the exchange of API calls between intermediary computing system 104 and Prepay Analysis Tool 200 to communicate claims data and/or initiate the prepay analysis process.

At step 306, an augmented or modified electronic health care claim is received from Prepay Analysis Tool 200. In particular, Prepay Analysis Tool 200 may perform a prepay analysis on the electronic health care claim and may return, to intermediary computing system 104, either an edited health care claim or a claim that has been appended with a code (e.g., modifier) associated with the prepay analysis. In some implementations, Prepay Analysis Tool 200 modifies one or more fields or data of the claim based on the results of the prepay analysis and returns the modified claim to intermediary computing system 104. For example, Prepay Analysis Tool 200 may edit pricing or claims information. In some implementations, Prepay Analysis Tool 200 appends a unique code to the claim before returning to intermediary computing system 104. The unique code may indicate results of the prepay analysis. For example, the code may be a link to additional information about the prepay analysis or may otherwise be usable by the recipient of the claim (e.g., a health care payor) to retrieve the results of the analysis. In other implementations, Prepay Analysis Tool 200 does not edit the claim itself, but instead send results of the prepay analysis (e.g., a report, the unique code) to intermediary computing system 104, whereupon intermediary computing system 104 modifies the claim. In yet other implementations, Prepay Analysis Tool 200 may store results (e.g., a report) of the prepay analysis (e.g., in database 216) which can be remotely accessed by an external party (e.g., payor computing system 106) based on the unique code, via an embedded link, or by accessing an account maintained by Prepay Analysis Tool 200.

At step 308, the augmented electronic health care claim is transmitted (e.g., forwarded) to a recipient computer. Generally, the recipient computer may be a computing device operated by a health care payor (e.g., an insurance company), such as payor computing system 106; however, it should be appreciated that other entities may be the recipient of claims. In any case, the electronic health care claim generally includes the above-mentioned unique code relating to the prepay analysis or otherwise indicates that the prepay analysis has been completed (and, accordingly, results are available). In this manner, the recipient computer receives the claim in the standard claim format (e.g., EDI 837), which reduces computational overheard. The recipient computer can then use the code to determine information related to the prepay analysis. For example, the recipient computer can use the code to look up or access a prepay analysis report or edits. Additionally, or alternatively, a report relating to the prepay analysis can be sent to the recipient computer.

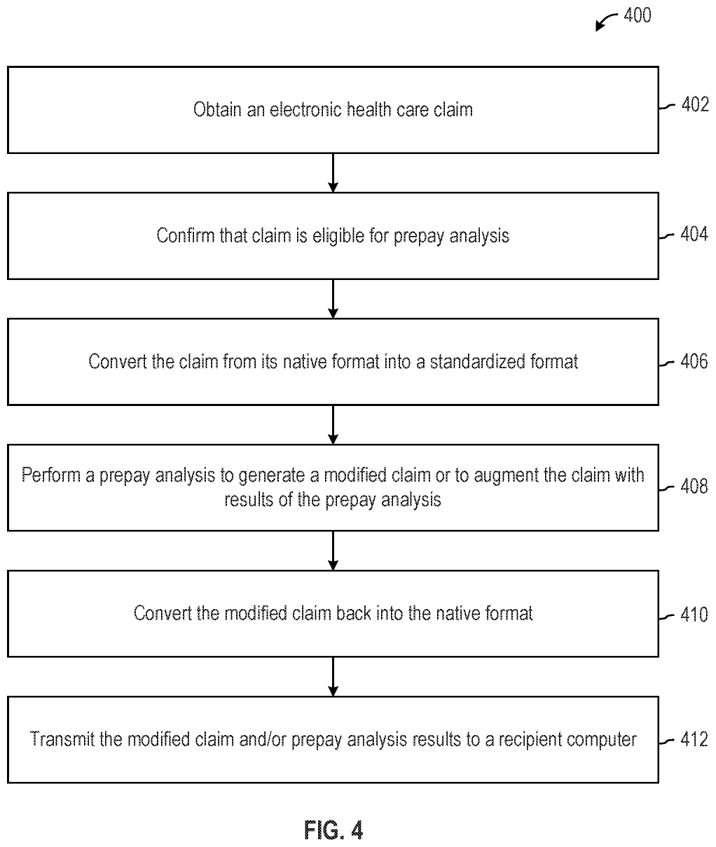

Referring now to FIG. 4, a flow chart of a process 400 for performing a prepay analysis on an electronic health care claim is shown, according to some implementations. In some implementations, process 400 is implemented by Prepay Analysis Tool 200, as described above; although, it will be appreciated that process 400, or certain steps of process 400, may be performed by intermediary computing system 104 or another device. It will be appreciated that certain steps of process 400 may be optional and, in some implementations, process 400 may be implemented using less than all of the steps. It will also be appreciated that the order of steps shown in FIG. 4 is not intended to be limiting.

At step 402, an electronic health care claim is obtained (e.g., by Prepay Analysis Tool 200). In some implementations, the electronic health care claim is received from intermediary computing system 104. For example, intermediary computing system 104 may forward the electronic health care claim to Prepay Analysis Tool 200 upon receipt from provider computing system 102. In some implementations, the electronic health care claim is received directly from provider computing system 102 or another entity. Generally, the electronic health care claim is in EDI 837 format.

At step 404, it is confirmed (e.g., verified) that the electronic health care claim is eligible for prepay analysis. In particular, Prepay Analysis Tool 200 may evaluate the claim to determine prepay analysis eligibility. In some implementations, an eligibility determination is made based on the intended recipient of the claim. For example, a payor ID (e.g., contained within the claim or determined based on the intended recipient of the claim) can be used to confirm whether the payor utilizes the prepay analysis service. In some implementations, determining whether a claim is eligible for prepay analysis includes querying a database of known payors having a previously established prepay review agreement (e.g., with the owner/operator of Prepay Analysis Tool 200) using the identifier to identify a match to one of the payors.

As mentioned above, for a claim to be eligible for prepay analysis, the payor (e.g., claim recipient) and/or provider (e.g., claim submitter) may be required to have subscribed to a prepay analysis service (e.g., offered by an entity that operates intermediary computing system 104 and/or Prepay Analysis Tool 200) or otherwise contracted with the entity that operates Prepay Analysis Tool 200. In other words, prepay analysis eligibility is often dependent on the intended recipient of the claim (e.g., a healthcare payor). Accordingly, in some implementations, step 404 further includes retrieving, from database 216, parameters for the prepay analysis associated with the identified payor/recipient. In other implementations, health care claims may be marked or otherwise identified for prepay analysis. For example, a health care claim may include a code or indicator which prompts Prepay Analysis Tool 200 to perform the prepay analysis.

At step 406, the electronic health care claim is optionally converted from its native data format into a standardized data format. In other words, the electronic health care claim can be converted to a format suitable for prepay analysis. In some implementations, this conversion includes converting the claim from a first data format into a second data format. In some implementations, the conversion process includes extracting data from one or more fields of the electronic health care claim. In many cases, but not exclusively, electronic health care claims are received in EDI 837 format. For example, in some cases, claims are received as in pipe delimited, XML, or other proprietary formats. Accordingly, electronic health care claims may be converted into a standard format for processing, such as EDI 837 or another format.

At step 408, a prepay analysis is performed on the electronic health care claim and, in some implementations, based on the results of the prepay analysis, a modified electronic health care claim is generated or the electronic health care claim is augmented based on the results of the analysis. As mentioned above, the prepay analysis may include one or more different reviews, checks, edits, or services as dictated by an agreement between the entity that owns/operates Prepay Analysis Tool 200 and the entity that requests the prepay analysis (e.g., the payor). In some implementations, Prepay Analysis Tool 200 modifies one or more fields or data of the claim based on the results of the prepay analysis and returns the modified claim to intermediary computing system 104. For example, Prepay Analysis Tool 200 may edit pricing or claims information. In some implementations, Prepay Analysis Tool 200 augments the claim by append a unique code to the claim before returning to intermediary computing system 104. The unique code may indicate results of the prepay analysis. For example, the code may be a link to additional information about the prepay analysis or may otherwise be usable by the recipient of the claim (e.g., a health care payor) to retrieve the results of the analysis. In some implementations, Prepay Analysis Tool 200 indicates that the prepay analysis was completed (e.g., via the appended code) and that results of the analysis are available for viewing/download. In some implementations, Prepay Analysis Tool 200 retains results of the prepay analysis for remote access by external computing devices (e.g., payor computing system 106). In some implementations, Prepay Analysis Tool 200 augments a data structure associated with the electronic health care claim based on the results of the prepay analysis, rather than modifying the claim itself. Subsequently, at step 410, the modified electronic health care claim is optionally converted back into its native data format.

At step 412, the modified or augmented electronic health care claim is transmitted to a recipient computer. In some implementations, the “recipient” computer is intermediary computing system 104. In some such implementations, intermediary computing system 104 may optionally perform one or more additional analyses or edits of the modified electronic health care claim and may then forward the modified claim to payor computing system 106 or the intended recipient. In other implementations (e.g., as illustrated in FIG. 1B), Prepay Analysis Tool 200 may transmit the electronic health care claim directly to payor computing system 106 or the intended recipient.

### Claim Status Integration

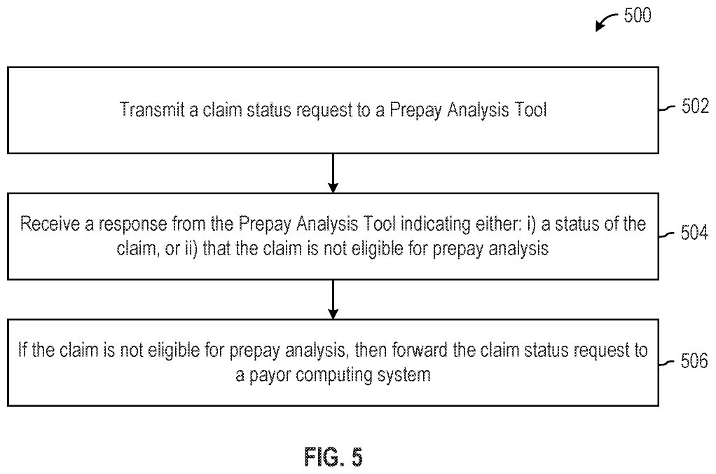

In some implementations, a secondary transaction for checking the status of submitted health care claims can be implemented by system 100, as described above. Claims status integration allows the submitter of a claim (e.g., a health care provider) or another entity to determine a status of their claim. For example, claims may occasionally be retained by one of intermediary computing system 104 or Prepay Analysis Tool 200 as prepay eligibility determinations and/or prepay analyses are performed; but, in some cases, only a payor (e.g., the claim recipient) has a contract in place to use Prepay Analysis Tool 200. Accordingly, intermediary computing system 104 and/or Prepay Analysis Tool 200 may implement a claims status checking process whereby a provider or another entity can request an updated claim status. In some implementations, a claim status check follows a similar communication path as described above with respect to FIG. 1A for claims; however, the claims status check process may be better understood with respect to FIG. 5, described below.

Referring now to FIG. 5, a flow chart of a process 500 for determine a status of an electronic health care claim is shown, according to some implementations. In some implementations, process 500 is implemented by intermediary computing system 104, as described above; although, it will be appreciated that process 500, or certain steps of process 500, may be performed by Prepay Analysis Tool 200. It will be appreciated that certain steps of process 500 may be optional and, in some implementations, process 500 may be implemented using less than all of the steps. It will also be appreciated that the order of steps shown in FIG. 5 is not intended to be limiting.

At step 502, a claim status request is transmitted to Prepay Analysis Tool 200. In some implementations, the request is transmitted by intermediary computing system 104 upon receipt from provider computing system 102 or another entity. In some implementations, any entity can request a claim status update if they have sufficient permissions within Prepay Analysis Tool 200. For example, Prepay Analysis Tool 200 may be accessible to any number of computing devices via API 218, in which case a provider could request a status update from a computer device that is not provider computing system 102 (e.g., a general purpose computer, a tablet, a smartphone, etc.). Generally, the status request identifies the claim based on, for example, a claim ID number.

At step 504, a response is received from Prepay Analysis Tool 200 which indicates either a status of the claim or that the claim is not eligible for prepay analysis. In some implementations, Prepay Analysis Tool 200 may first determine whether the claim or an intended recipient of the claim is eligible for prepay analysis. If so, Prepay Analysis Tool 200 may determine if the claim is with Prepay Analysis Tool 200 and/or is undergoing prepay analysis. Prepay Analysis Tool 200 may then return a status of the claim (e.g., “undergoing prepay analysis”). If, however, the claim is not eligible for prepay analysis or if the claim is no longer with Prepay Analysis Tool 200, then Prepay Analysis Tool 200 may return an appropriate indication.

At step 506, the claim status request is forwarded to payor computing system 106 or the original recipient of the claim if it is determined that the claim was not eligible for prepay analysis and/or that the claim is no longer with Prepay Analysis Tool 200 (e.g., because the prepay analysis is completed). Receiving the claim status request may prompt payor computing system 106 to respond with a claims status. In some implementations, payor computing system 106 may return a claims status to intermediary computing system 104, which forwards the claim status to provider computing system 102 or the entity that requested the status update.

Alternatively, if it is determined at step 504 that the claim was eligible for prepay analysis and that the claim is currently with Prepay Analysis Tool 200, then intermediary computing system 104 may return the status to the requestor.

### Configuration of Certain Implementations

The construction and arrangement of the systems and methods as shown in the various implementations are illustrative only. Although only a few implementations have been described in detail in this disclosure, many modifications are possible (e.g., variations in sizes, dimensions, structures, shapes and proportions of the various elements, values of parameters, mounting arrangements, use of materials, colors, orientations, etc.). For example, the position of elements may be reversed or otherwise varied, and the nature or number of discrete elements or positions may be altered or varied. Accordingly, all such modifications are intended to be included within the scope of the present disclosure. The order or sequence of any process or method steps may be varied or re-sequenced according to alternative implementations. Other substitutions, modifications, changes, and omissions may be made in the design, operating conditions, and arrangement of the implementations without departing from the scope of the present disclosure.

The present disclosure contemplates methods, systems, and program products on any machine-readable media for accomplishing various operations. The implementations of the present disclosure may be implemented using existing computer processors, or by a special purpose computer processor for an appropriate system, incorporated for this or another purpose, or by a hardwired system. Implementations within the scope of the present disclosure include program products including machine-readable media for carrying or having machine-executable instructions or data structures stored thereon. Such machine-readable media can be any available media that can be accessed by a general purpose or special purpose computer or other machine with a processor. By way of example, such machine-readable media can comprise RAM, ROM, EPROM, EEPROM, CD-ROM or other optical disk storage, magnetic disk storage or other magnetic storage devices, or any other medium which can be used to carry or store desired program code in the form of machine-executable instructions or data structures, and which can be accessed by a general purpose or special purpose computer or other machine with a processor.

When information is transferred or provided over a network or another communications connection (either hardwired, wireless, or a combination of hardwired or wireless) to a machine, the machine properly views the connection as a machine-readable medium. Thus, any such connection is properly termed a machine-readable medium. Combinations of the above are also included within the scope of machine-readable media. Machine-executable instructions include, for example, instructions and data which cause a general-purpose computer, special purpose computer, or special purpose processing machines to perform a certain function or group of functions.

Although the figures show a specific order of method steps, the order of the steps may differ from what is depicted. Also, two or more steps may be performed concurrently or with partial concurrence. Such variation will depend on the software and hardware systems chosen and on designer choice. All such variations are within the scope of the disclosure. Likewise, software implementations could be accomplished with standard programming techniques with rule-based logic and other logic to accomplish the various connection steps, processing steps, comparison steps and decision steps.

It is to be understood that the methods and systems are not limited to specific synthetic methods, specific components, or to particular compositions. It is also to be understood that the terminology used herein is for the purpose of describing particular implementations only and is not intended to be limiting.

As used in the specification and the appended claims, the singular forms “a,” “an” and “the” include plural referents unless the context clearly dictates otherwise. Ranges may be expressed herein as from “about” one particular value, and/or to “about” another particular value. When such a range is expressed, another implementation includes from the one particular value and/or to the other particular value. Similarly, when values are expressed as approximations, by use of the antecedent “about,” it will be understood that the particular value forms another implementation. It will be further understood that the endpoints of each of the ranges are significant both in relation to the other endpoint, and independently of the other endpoint.

“Optional” or “optionally” means that the subsequently described event or circumstance may or may not occur, and that the description includes instances where said event or circumstance occurs and instances where it does not.

Throughout the description and claims of this specification, the word “comprise” and variations of the word, such as “comprising” and “comprises,” means “including but not limited to.” and is not intended to exclude, for example, other additives, components, integers, or steps. “Exemplary” means “an example of” and is not intended to convey an indication of a preferred or ideal implementation. “Such as” is not used in a restrictive sense, but for explanatory purposes.

Disclosed are components that can be used to perform the disclosed methods and systems. These and other components are disclosed herein, and it is understood that when combinations, subsets, interactions, groups, etc. of these components are disclosed that while specific reference of each various individual and collective combinations and permutation of these may not be explicitly disclosed, each is specifically contemplated and described herein, for all methods and systems. This applies to all aspects of this application including, but not limited to, steps in disclosed methods. Thus, if there are a variety of additional steps that can be performed it is understood that each of these additional steps can be performed with any specific implementation or combination of implementations of the disclosed methods.

What is claimed is:

1. A system comprising:
a processor; and
memory having instructions stored thereon that, when executed by the processor, cause the system to:
obtain an electronic health care claim;
determine whether the electronic health care claim is eligible for prepay analysis based on an identifier for an intended recipient of the electronic health care claim;
if it is determined that the electronic health care claim is eligible for the prepay analysis, then: i) perform the prepay analysis, and ii) augment the electronic health care claim based on results of the prepay analysis; and
transmit the augmented electronic health care claim to a recipient computer associated with the intended recipient.

2. The system of claim 1, wherein obtaining the electronic health care claim comprises intercepting the electronic health care claim as it is transmitted from a sender computer to the recipient computer.

3. The system of claim 2, wherein the processor and the memory are components of a server operated by a clearinghouse.

4. The system of claim 1, wherein determining whether the electronic health care claim is eligible for prepay analysis comprises:
querying a database of known health care payors having a previously established prepay review agreement using the identifier to identify a match to one of the known health care payors; and
retrieving, from the database, parameters for the prepay analysis associated with the one of the known health care payors.

5. The system of claim 1, wherein augmenting the electronic health care claim comprises appending a unique code to the electronic health care claim.

6. The system of claim 5, wherein the unique code provides access to a report of the prepay analysis.

7. The system of claim 6, wherein the unique code comprises a selectable link which, when selected by a user, causes a user device to navigate to the report of the prepay analysis.

8. The system of claim 1, wherein the prepay analysis comprises one or more of a records review, claims code editing, coordination of benefits, and pricing verification.

9. The system of claim 1, wherein the instructions further cause the system to:
convert the electronic health care claim from a native data format into a standardized data format prior to determining whether the electronic health care claim is eligible for prepay analysis; and
convert the modified electronic health care claim back to the native data format prior to transmitting the modified electronic health care claim to the recipient computer.

10. The system of claim 1, wherein the instructions further cause the system to:
receive a status request for the electronic health care claim;
determine a status of the electronic health care claim if the electronic health care claim is determined to be eligible for prepay analysis; and
return an indication of the status.

11. A method comprising:
obtaining an electronic health care claim;
determining whether the electronic health care claim is eligible for prepay analysis based on an identifier for an intended recipient of the electronic health care claim;
if it is determined that the electronic health care claim is eligible for the prepay analysis, then i) performing the prepay analysis, and ii) augmenting the electronic health care claim based on results of the prepay analysis; and
transmitting the augmented electronic health care claim to a recipient computer associated with the intended recipient.

12. The method of claim 11, wherein obtaining the electronic health care claim comprises intercepting the electronic health care claim as it is transmitted from a sender computer to the recipient computer.

13. The method of claim 11, wherein determining whether the electronic health care claim is eligible for prepay analysis comprises:
querying a database of known health care payors having a previously established prepay review agreement using the identifier to identify a match to one of the known health care payors; and
retrieving, from the database, parameters for the prepay analysis associated with the one of the known health care payors.

14. The method of claim 11, wherein augmenting the electronic health care claim comprises appending a unique code to the electronic health care claim.

15. The method of claim 14, wherein the unique code provides access to a report of the prepay analysis.

16. The method of claim 15, wherein the unique code comprises a selectable link which, when selected by a user, causes a user device to navigate to the report of the prepay analysis.

17. The method of claim 11, wherein the prepay analysis comprises one or more of a records review, claims code editing, coordination of benefits, and pricing verification.

18. The method of claim 11, further comprising:
converting the electronic health care claim from a native data format into a standardized data format prior to determining whether the electronic health care claim is eligible for prepay analysis; and
converting the modified electronic health care claim back to the native data format prior to transmitting the modified electronic health care claim to the recipient computer.

19. The method of claim 11, further comprising:
receiving a status request for the electronic health care claim;
determining a status of the electronic health care claim if the electronic health care claim is determined to be eligible for prepay analysis; and
returning an indication of the status.

20. A non-transitory computer readable medium having instructions stored thereon that, when executed by a processor, cause a device to:
obtain an electronic health care claim;
determine whether the electronic health care claim is eligible for prepay analysis based on an identifier for an intended recipient of the electronic health care claim;
if it is determined that the electronic health care claim is eligible for the prepay analysis, then: i) perform the prepay analysis, and ii) augment the electronic health care claim with a unique code based on results of the prepay analysis; and
transmit the augmented electronic health care claim to a recipient computer associated with the intended recipient.
